(19) United States
(12) Patent Application Publication
(10) Pub. No.: US 2016/0258028 A1
(43) Pub. Date: Sep. 8, 2016

(54) METHODS AND COMPOSITIONS FOR DETECTING COLORECTAL CANCER USING MICRO RNAs

(71) Applicant: University of Louisville Research Foundation, Inc., Louisville, KY (US)

(72) Inventors: Susan Galandiuk, Louisville, KY (US); Ziad Kanaan, Dearborn, MI (US); Maurice-Robert Eichenberger, Louisville, KY (US)

(21) Appl. No.: 15/062,419

(22) Filed: Mar. 7, 2016

Related U.S. Application Data
(62) Division of application No. 13/870,171, filed on Apr. 25, 2013, now abandoned.
(60) Provisional application No. 61/639,646, filed on Apr. 27, 2012.

(51) Int. Cl. C12Q 1/68

(52) U.S. Cl. CPC C12Q 1/6886; C12Q 2600/178; C12Q 2600/16

(57) ABSTRACT

Methods of detecting or predicting the presence of CRC based on the amounts of particular miRNAs in plasma or tissue are described.

### CROSS-REFERENCE TO RELATED APPLICATIONS

This application is a Divisional of, and claims the benefit of priority under 35 U.S.C. §121 to, U.S. application Ser. No. 13/870,171 filed Apr. 25, 2013, which claims the benefit of priority under 35 U.S.C. §119(e) to U.S. Application No. 61/639,646 filed Apr. 27, 2012.

### TECHNICAL FIELD

This disclosure generally relates to microRNA (miRNA) and its use in screening and detection of cancer.

### BACKGROUND

Colorectal cancer (CRC) is the third-most common cancer worldwide, second only to lung cancer. In 2011, it was estimated that about 141,210 new cases of CRC were diagnosed, and 49,380 deaths resulted in the US from the disease. The prognosis of CRC is associated with stage-at-diagnosis and is curable if detected early; 5-year survival rates range from >93% for stage I disease to <8% for stage IV disease. However, the challenge is that many patients are asymptomatic in early stages. Therefore, there is a need for an accurate, non-invasive biomarker to identify CRC earlier.

Currently, CRC is primarily diagnosed through colonoscopy, a procedure that is expensive, requires bowel preparation, sedation, and may also be associated with medical complications. Many patients hesitate or completely avoid having a colonoscopy due to its invasive nature. Less-invasive diagnostic methods such as carcinoembryonic antigen (CEA) screening in blood have less-than-ideal sensitivity and specificity (74% and 87%, respectively). Therefore, a need exists for a more accurate and precise blood test that can diagnose CRC earlier.

### SUMMARY

The present disclosure describes methods of detecting or predicting the presence of CRC based on the amounts of particular miRNAs in plasma or tissue.

In one aspect, a method of screening an individual for the presence or absence of sporadic colorectal cancer (CRC) is provided. Such a method typically includes providing a biological sample from the individual, wherein the biological sample is blood or plasma; and determining the level of miR-21 in the biological sample. Generally, a higher level of miR-21 relative to the level of miR-21 in a control sample is indicative of the presence of sporadic CRC in the individual.

In one embodiment, the individual is at a higher risk for CRC, has a personal history of CRC or polyps, or has a family history of sporadic CRC or polyps. In one embodiment, the level of miR-21 is determined using RT-PCR. In one embodiment, the level of miR-21 is normalized using housekeeping miRNA U6B. In one embodiment, the method provides a sensitivity of at least 90%, a specificity of at least 90%, and a ROC having an AUC of >0.9.

In another aspect, a method of screening an individual for the presence or absence of sporadic colorectal cancer (CRC) is provided. Such a method typically includes providing a biological sample from the individual, wherein the biological sample is a tissue sample; and determining the levels of at least four miRNAs in the biological sample. Generally, the at least four miRNAs comprises miR-31, miR-135b, miR-1 and miR-133a, wherein a higher level of miR-31 and miR-135b and a lower level of miR-1 and miR-133a relative to corresponding miRNA levels in a control sample is indicative of the presence of sporadic CRC in the individual.

In one embodiment, the individual is at a higher risk for CRC, has a personal history of CRC or polyps, or has a family history of sporadic CRC or polyps. In one embodiment, the tissue sample is selected from the group consisting of colon tissue and rectal tissue. In one embodiment, the levels of the at least four miRNAs are normalized using housekeeping miRNA U6B. In one embodiment, the levels of the at least four miRNAs are determined using RT-PCR. In one embodiment, the method provides a sensitivity of at least 95% (e.g., at least 99%, 100%). a specificity of at least 85%, and ROC having an AUC of >0.9.

In yet another aspect, an article of manufacture is provided. Such an article of manufacture (e.g., a kit) typically includes a pair of miR-31 amplification primers; a pair of miR-135b amplification primers; a pair of miR-1 amplification primers; and a pair of miR-133a amplification primers.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a flow chart of the experiments using tissue.

FIG. 2 is a flow chart of the experiments using plasma.

FIG. 3A shows the receiver operating characteristic (ROC) curves for miR-31, miR-135b, miR-1, and miR-133a combined in the “training” cohort (30 sporadic CRC tissues+30 adjacent normal tissues).

FIG. 3B shows the ROC curves for miR-31, miR-135b, miR-1, and miR-133a combined in the “test” cohort (16 new sporadic CRC tissues+16 adjacent normal tissues). Area under the curve (AUC) values were >0.9 in both groups (red circles), indicating excellent discriminative power.

FIG. 4A shows the ROC curve for miR-21 in the plasma “training” group (30 plasma samples from the same CRC patients from which tissue samples were obtained+30 healthy subjects in comparison group (CG1)).

FIG. 4B shows the ROC curve for miR-21 in the plasma “test” group (20 plasma samples from new patients+20 healthy subjects in comparison group 2 (CG2)). The AUC value for miR-21 in the “test” group was high, indicating excellent discriminatory power.

### DETAILED DESCRIPTION

miRNAs are small, non-coding RNA molecules that regulate gene expression post-transcriptionally by specifically binding to the 3′ untranslated regions (3′ UTRs) of target messenger RNAs (mRNA). Previous studies of miRNAs in CRC have provided inconsistent results due to the use of variable detection methods, different source materials, and the difficulty in determining an appropriate reference (e.g., housekeeping) miRNA. These problems have been addressed, as described herein, through, at least in part, the development of a reproducible, sensitive, and specific miRNA extraction procedure. The methods described herein can be used to reliably obtain and measure differential miRNA expression in tissue or plasma samples. The methods described herein using the miRNA biomarkers identified herein result in higher sensitivity and specificity for CRC than currently used biomarkers. The results described herein may lead to a clinical practice paradigm shift away from traditional colonoscopy for cancer screening.

Methods of screening an individual for the presence or absence of sporadic colorectal cancer (CRC; e.g., early-stage CRC) are described herein. In one embodiment, a biological sample of blood or plasma is provided from the individual and the level of miR-21 is determined. As demonstrated herein, a higher level of miR-21, relative to the level of miR-21 in a control sample, indicates or is predictive of the presence of sporadic CRC in the individual. In some instances, the level of miR-21 is statistically significantly higher than the level in the control sample. In fact, the diagnostic efficiency of miR-21 in plasma may be near-optimal for discriminating CRC.

In another embodiment, a biological tissue sample is provided from the individual. The sample can be from, for example, colon tissue or rectal tissue. In this embodiment, the level of at least four miRNAs is determined. The at least four miRNAs includes miR-31, miR-135b, miR-1 and miR-133a, and, as described herein, a higher level of miR-31 and miR-135b and a lower level of miR-1 and miR-133a relative to the corresponding miRNA level in a control sample indicates or is predictive of the presence of sporadic CRC in the individual.

Although not intended to be limiting, individuals that might benefit from the screening methods disclosed herein include those individuals that are at a higher risk for CRC (e.g., individuals greater than 50 years old, smokers, African-Americans, and/or individuals that eat a diet low in fiber), individuals that have a personal history of CRC or polyps, or individuals that have a family history of sporadic CRC or polyps.

Methods of determining the level of one or more miRNAs are well known in the art. For example, the level of miRNAs can be determined using routine RT-PCR. Simply by way of example, total RNA can be extracted from raw serum (e.g., 0.5 mL of raw serum) using TRIzol® LS Reagent, reverse transcribed and amplified in the presence of a pair of miR-21 primers to produce cDNAs using, for example, a RT-PCR Kit from Life Technologies (Carlsbad, Calif.). In another example, total RNA can be extracted from colon or rectal tissue (e.g., 20 mg of tissue) using, for example, Ambion mirVana™ miRNA Isolation Kit (Life Technologies®), and reverse transcribed and amplified in the presence of pairs of miR-31, miR-135b, miR-1, and miR-133a primers, using, for example, the aforementioned RT-PCR kit from Life Technologies®. Following RT-PCR, the amount or level of the resulting amplified product can be determined using, for example, a spectrophotometer (e.g., a Nanodrop 2000 from Thermo Scientific® (Middlesex, Mass.)). Alternatively, other methods suitable for detecting and quantitating RNAs can be used such as Northern blotting following by phosphoimaging.

Oligonucleotide primers for use in detecting the indicated miRNAs (e.g., for use in RT-PCR reactions) can be readily designed by a person skilled in the art. Primers typically are designed using computer software (e.g., Vector NTI® (Life Technologies); Primer Premier (Premier Biosoft); OLIGO (Molecular Biology Insights)) and the sequence of the miRNA. For example, the sequence of miR-21 from Homo sapiens can be found in GenBank Accession No. NR 029493.1; the sequence of miR-31 from Homo sapiens can be found in GenBank Accession No. NR 029505.1; the sequence of miR-135b from Homo sapiens can be found in GenBank Accession No. NR_029893.1; the sequence of miR-1 from Homo sapiens can be found in GenBank Accession No. NR_029780.1; and the sequence of miR-133a from Homo sapiens can be found in GenBank Accession No. NR_029675.1.

In order to accurately determine the levels of one or more miRNAs in a biological sample and to be able to reliably compare those levels to the corresponding levels in a control sample (i.e., a biological sample known to be free of CRC), means for normalizing the samples to one another is required. One of the significant improvements of the methods described herein is the ability to obtain stable and readily quantitatable amounts of the housekeeping miRNA, U6B (Chen et al., 2005, “Real-time quantification of microRNAs by stem-loop RT-PCR”, Nuc. Acids Res., 33:e179). The ability to obtain consistent and reproducible amounts of U6B improved the accuracy and, hence, the statistical significance of the methods and markers described herein, particularly given that current methods require adding synthetic miRNAs as an internal control. See, for example, Kanaan et al. (2012, Hum. Mutat., 33:551-60), Huang et al. (2010, Int. J. Cancer, 127:118-26), Koga et al. (2010, Cancer Prev. Res., 3:1435-42), Link et al. (2010, Cancer Epidem., 19:1766-74), Ng et al. (2009, Gut, 58:1375-81), and Pu et al. (2010, J. Gastroent. Hepat., 25:1674-80).

The RNA extraction method described herein is modified slightly from the TRIZOL LS reagent standard protocol of RNA extraction (tool.invitrogen.com/content/sfs/manuals/trizol_ls_reagent.pdf on the World Wide Web). For example, the initial centrifugation step is longer (20 mins instead of 10 mins) and faster (20 kG instead of 12 kG) than recommended. In addition, following several precipitations, the nucleic acid is allowed to dry for at least an hour, as a shorter drying time was determined to reduce the amount and viability of the isolated miRNA. In addition, the RT-PCR conditions were modified slightly from the manufacturers suggested protocol (Applied Biosystems). For example, the total volume was increased from 15 μl to 21 μl.

The methods and miRNA markers described herein resulted in very high sensitivity and specificity values for predicting or detecting the presence or absence of CRC in an individual. For example, using blood or plasma samples, the sensitivity of the screening methods described herein is 90% or greater than 90% (e.g., greater than 91%, greater than 92%) and the specificity of the screening methods described herein also is 90% or greater than 90% (e.g., greater than 91%, greater than 92%). In addition, using tissue samples, the sensitivity of the screening methods described herein is 95% or greater than 95% (e.g., greater than 99%, 100%) and the specificity of the screening methods described herein is 85% or greater than 85% (e.g., greater than 86%, greater than 87%). Further, the methods described herein resulted in AUCs of 0.9 or greater than 0.9 in ROC analyses, which indicates that the one or more miRNAs exhibit excellent discriminatory power for CRC.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Patients

The study described herein was approved by the University of Louisville Institutional Review Board (IRB), and written informed consent was obtained from all subjects. Patients were derived from a large university medical and surgical digestive disease practice and from the University of Louisville Surgical Biorepository database. Patients included those who were diagnosed with sporadic CRC. Diagnosis was confirmed using histopathology. A total of 66 patients with sporadic CRC were the focus of this study (Appendix I). Patients were excluded if they had any of the following: clinical diagnosis of familial adenomatous polyposis or hereditary non-polyposis CRC, or undergoing chemotherapy or radiotherapy.

### Example 2

### Tissue Experiments

CRC tissue and adjacent non-neoplastic tissue samples from each of 46 patients with sporadic CRC were used in the tissue experiments described herein. Tumors were staged according to the tumor-node-metastasis (TNM) staging system.

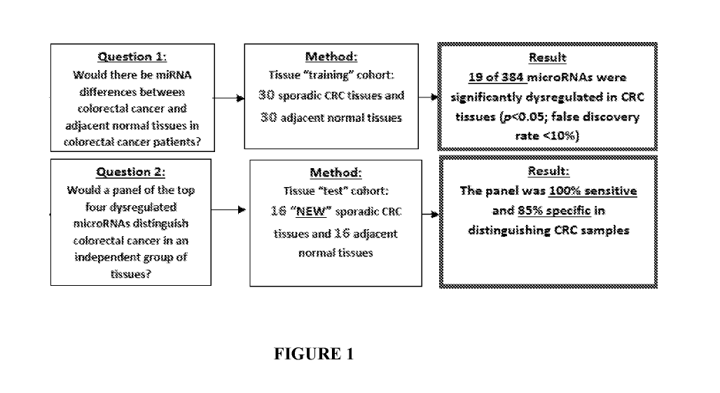

Differences in miRNA levels between CRC tissue and adjacent non-neoplastic tissue were first examined within each of the 30 enrolled patients in the “training” group (Appendix I and FIG. 1). This study was conducted using 60 frozen tissue samples taken from each of the enrolled patients (cancer and adjacent normal tissue from each patient). The miRNA findings from the “training” cohort were then validated in an independent “test” cohort of 16 patients with sporadic CRC (Appendix I and FIG. 1).

### Example 3

### Tissue RNA Extraction

Total tissue RNA was extracted using the Ambion mirVana™ miRNA Isolation Kit (Life Technologies®, Carlsbad, Calif.). Approximately 20 mg of tissue was used from each sample. The concentration and purity of the RNA sample were measured using the Nanodrop 2000 spectrophotometer (Thermo Scientific®, Middlesex, Mass.). All samples passed the quantity and purity checks as defined and stated in the protocol of the mirVana™ miRNA Isolation Kit.

### Example 4

### Tissue miRNA Quantification

The cDNA was obtained for each extracted RNA sample from the “training” cohort through reverse transcription using Megaplex® Reverse Transcription Human Pool A v2.1 (Life Technologies®, Carlsbad, Calif.), which contains a set of 384 stem-looped reverse transcription primers. Subsequently, the cDNA was loaded into a 384 miRNA TagMan® low-density array card (TLDA) (Applied Biosystems®). The TLDA cards were then run using Applied Biosystems Viia7® for the Real-Time Polymerase Chain Reaction (RT-PCR) and analysis.

### Example 5

### miRNA Extraction and Amplification Protocols

A plasma miRNA extraction method was developed and used, which consistently resulted in high levels of pure miRNA, including the housekeeping miRNA, U6B, useful as an internal reference. Initially, 75% EtOH, chloroform, H₂0, and 100% isopropyl alcohol were prepared and put on ice for future use. To begin the RNA extraction, 1 mL of Trizol LS, a phenol-guanidine isothiocyanate solution, was added to the plasma sample. The solution was mixed by pipetting up and down several times, centrifuged at 20 kG for 20 mins, and the top aqueous layer containing the miRNA was transferred to a new 1.5 mL tube.

The phenol-guanidine isothiocyanate then was removed using a chloroform extraction. 0.2 ml of ice-cold chloroform was added to the tube, mixed by numerous inversions, and incubated for 10 mins at 4° C. The sample was then centrifuged at 12 kG for 15 mins, and the top aqueous layer containing the miRNA was removed and transferred to a new 1.5 mL tube. During transfer, the middle layer was avoided by at least 0.5 cm to reduce the possibility of contamination.

The miRNAs were then precipitated. 0.5 mL of ice-cold 100% isopropyl alcohol was added, mixed by numerous inversions, and incubated for 10 mins at 4° C. The liquid was gently poured out of the tube and the open top was pressed on a paper towel to dry. 1 mL of ice-cold 75% EtOH was added, mixed by numerous inversions, and centrifuged at 7,500 G for 5 mins. Again, the liquid was gently poured out of the tube and the open top was pressed on a paper towel to dry. The tube was air dried in an inverted position for at least an hour. 50 μL RNase-free H₂0 was added and mixed by pipetting. The tube was incubated on ice for 20 mins, and the resulting RNA was nano-dropped to quantitate.

The PCR reaction was modified from the manufacturers suggested protocol (Applied Biosystems) as follows: 9 μl of Master Mix (instead of 7 μl), 5 μl of primers (instead of 3 μl) (see, for example, tools.invitrogen.com/content/sfs/manuals/cms_084553.pdf on the World Wide Web), 7 μl of sample (instead of 5 μl), and 21 μl reaction (instead of 15 μl; reaction=TAQMAN Advanced Fast Master Mix (2×), TAQMAN Gene Expression Assay (20×), cDNA template and nuclease-free water) were used. The amplification protocol included a 2 min hold for UNG incubation, 20 second hold for polymerase activation, followed by 40 cycles of PCR denaturing (3 sec) and annealing (30 sec).

### Example 6

### Validation in Tissue Cohort

The four most dysregulated miRNAs [p<0.05, False Discovery Rate (FDR): 10%] were then validated in a second blinded “test” cohort of 16 CRC patients (Appendix I and FIG. 1). For each sample, 10 ng of RNA was reverse transcribed into cDNA using MultiScribe™ reverse transcriptase and gene-specific stem-loop primers, which target the mature miRNA sequence (miR-31, miR-135b, miR-1, miR-133a, and RNU6B). RT-PCR was performed using Taqman® probes that bind to a complementary sequence in the target gene between the forward and reverse primers. The RT-PCR reactions were performed on a Step-One Plus® Fast RT-PCR System (Applied Biosystems®) using default thermal cycling conditions. All reactions were performed in duplicate. The threshold and standard deviation accepted for intra- and inter-assay replicates was 0.3. Raw fluorescence data (Ct values) generated by the RT-PCR instrument were exported and normalized to RNU6B as an endogenous control miRNA to generate relative quantities.

### Example 7

### Plasma Experiments

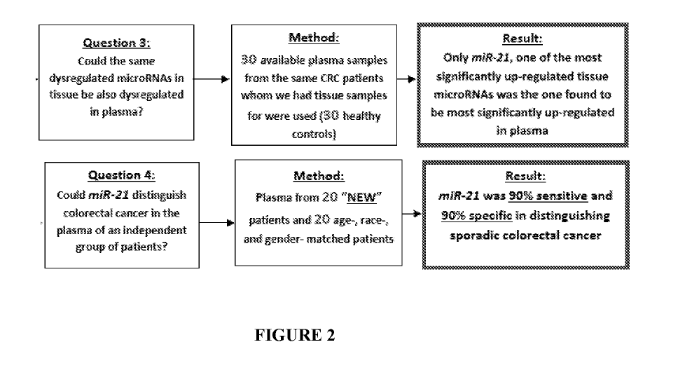

Thirty available plasma samples from the same CRC patients that provided tissue samples were used as the plasma “training” cohort. Plasma from 30 healthy subjects was used in the first comparison group (CG1) as a control for the plasma “training” cohort (Appendix I and FIG. 2).

The findings in the plasma “training” cohort were then validated in a plasma “test” cohort. This group consisted of 20 “new” CRC patients and 20 age-, gender-, and race-matched subjects in the second comparison group (CG2) (Appendix I and FIG. 2). Individuals in both comparison groups (CG1 & CG2) were patients with no known malignancy or active inflammatory condition and were seeking care in a sophisticated specialty practice. Their main complaints were related to constipation and their principal operations (if any) were anorectal procedures.

### Example 8

### Plasma RNA Extraction and miRNA Quantification

A total of 0.25 mL of raw serum was used for RNA extraction from each patient sample. The protocol from Ambion® for the TRIzol® LS Reagent was modified as described above in Example 5 and consistently yielded pure and high plasma miRNA levels with a stable RNU6B housekeeping miRNA for reference. The RNA obtained from each sample was analyzed for quality purposes as described above. The same miRNA quantification protocol that was used in the tissue validation section above was used for serum. The three most up-regulated (miR-31, miR-135b, and miR-21) and the three most down-regulated miRNAs (miR-1, miR-133a, and miR-133b) from the tissue experiments were studied.

### Example 9

### Validation in Plasma Test Cohort

The significantly dysregulated miRNAs from the plasma “training” cohort was then validated in a different “test” cohort using single miRNA Taqman® primers (FIG. 2). Similar procedure for miRNA extraction and quantification was used as described above. RNU6B was also used as the housekeeping miRNA for normalization.

### Example 10

### Statistical Analysis

The paired t-tests for the difference of miRNA levels between CRC tissues and normal tissues were used to find the significantly up- or down-regulated miRNAs. Multiple test control was based on controlling the FDR at 10% level. A logistic regression model was established using the two top up-regulated and two down-regulated miRNAs, which was used for predicting the cancer and normal group for the validation data. Sensitivity and specificity for this prediction were calculated. The receiver operator characteristic (ROC) curves with AUC values were generated using current versions of SAS (The NC, S. SA, V. Inc. 2003; 9) and R (R., Core A, Team R, “Language and Environment for Statistical Computing”, Statistical Computing, Vienna, Austria, 2005; M. G. “Receiver Operating Characteristics (ROC) Curves”, Proceedings of the Thirty-first Annual SAS Users Group International Conference, Cary, N.C., SAS Institute Inc., Paper 210-31; 120.2006; and Rai et al., “Statistical Analysis of Repeated MicroRNA High Throughput Data with Application to Human Heart Failure: A Methodology Review”, Open Access Medical Statistics, 2012, In Press).

### Example 11

### Findings in Tissue Experiments

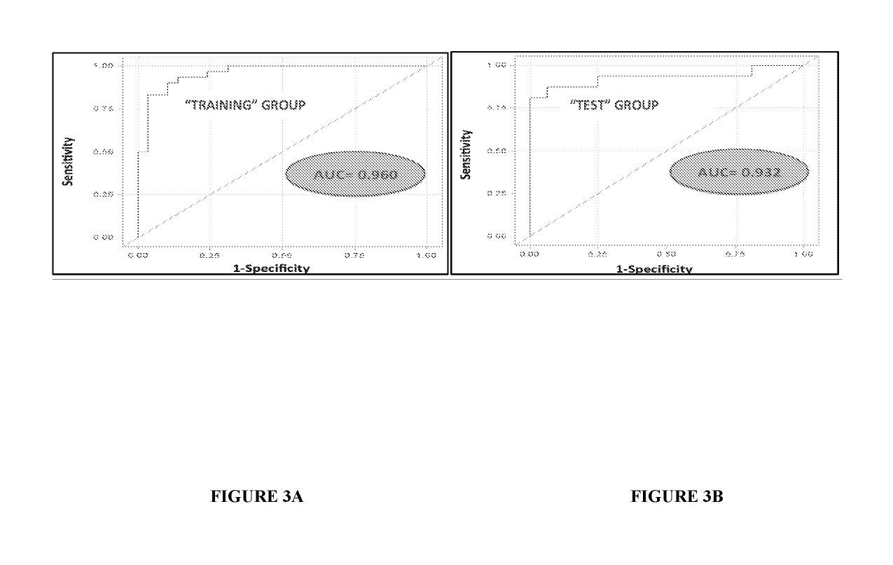

Nineteen of 380 miRNAs were dysregulated in CRC tissue in the “training” cohort (p<0.05, FDR: 10%) (Tables 1A and 1B). A combined panel of the two most up-regulated (miR-31; miR-135b) and two most down-regulated (miR-1; miR-133a) miRNAs identified CRC in the test cohort with 100% sensitivity and 85% specificity. miR-31 was more up-regulated in stages III & IV compared to stages I & II (p<0.05). The ROC curve for the four-miRNA panel in tissue showed a significantly high area under the curve (AUC) of 0.960 in the “training” group and 0.932 in the “test” group (FIGS. 3A and 3B). Both of these values were >0.9, indicating excellent discriminative power (Swets, “Measuring the Accuracy of Diagnostic Systems”, Science, 1988, 240(4857):1285-1293).

TABLE 1A
Ten most significantly up-regulated miRNAs (p < 0.05)

| Up-regulated miRNAs | Fold Change |
|---|---|
| miR-31* | 8.42 |
| miR-135b* | 7.08 |
| miR-21* | 3.42 |
| miR-183 | 3.32 |
| miR-886 | 2.92 |
| miR-301b | 2.59 |
| miR-708 | 2.18 |
| miR-493 | 2.04 |
| miR-382 | 2.03 |
| miR-362 | 1.73 |

TABLE 1B
Nine most significantly down-regulated miRNAs (p < 0.05)

| Down-regulated miRNAs | Fold Change |
|---|---|
| miR-1* | −4.35 |
| miR-133a* | −4.17 |
| miR-133b | −3.23 |
| miR-204 | −3.13 |
| miR-145 | −2.94 |
| miR-9 | −2.86 |
| miR-139-5p | −2.70 |
| miR-138 | −2.70 |
| miR-885-5p | −2.38 |

*tested in plasma training group

### Example 12

### Findings in Plasma Experiments

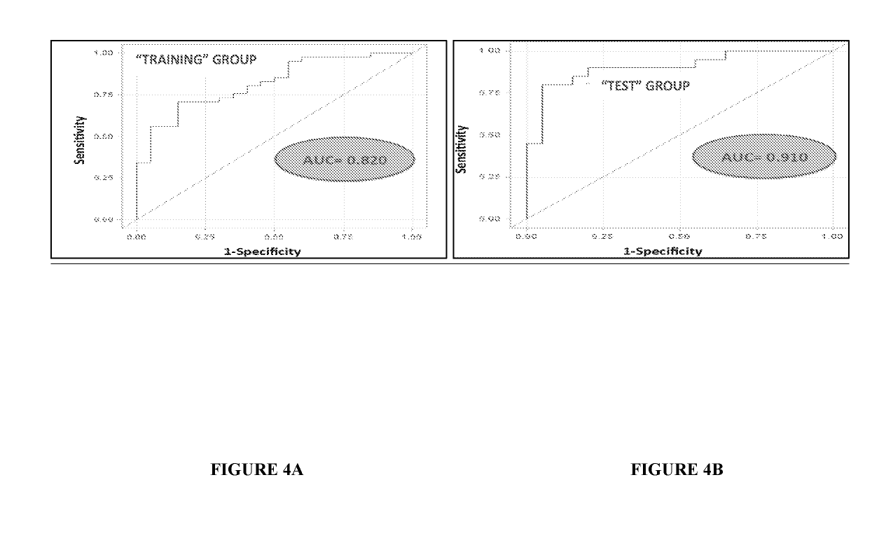

The validated four-microRNA panel in tissue along with miR-21 (the third-most up-regulated miRNA in tissue and a common plasma miRNA involved in multiple cancers) and miR-133a (the third most down-regulated miRNA) were evaluated in the plasma “training” group. Only miR-21, the third most up-regulated tissue miRNA, differentiated CRC patients from controls with 90% specificity and sensitivity in the plasma “training” cohort. Only miR-21 was further validated in an independent plasma “test” cohort. This group consisted of 20 “new” CRC patients and 20 age-, gender-, and race-matched individuals in the comparison group (Table 2). The AUC value for miR-21 in the “test” group was >0.9 (FIGS. 4A and 4B) indicating excellent discriminatory power (Swets, supra).

TABLE 2
miRNA-21-fold changes in plasma of CRC patients¹

| Matched Pair | Fold Changes |
|---|---|
| 1 | 5.4 |
| 2 | 17.6 |
| 3 | 45.6 |
| 4 | 182.6 |
| 5 | 1274.2 |
| 6 | 306.9 |
| 7 | 0.5* |
| 8 | 279.2 |
| 9 | 9.5 |
| 10 | 0.12* |
| 11 | 4.1 |
| 12 | 3.0 |
| 13 | 32.9 |
| 14 | 362.5 |
| 15 | 48.6 |
| 16 | 3.5 |
| 17 | 12.3 |
| 18 | 1.9 |
| 19 | 27.2 |
| 20 | 324.1 |

¹compared to 20 age- gender-, and race-matched controls in the “test” group
*fold change > 1 denotes up-regulating of miR-21. Matched pairs 7 and 10 showed down-regulation of miR-21.
²FDR: false discovery rate

### Example 13

### Comparison of miRNA Panel to Reported miRNAs and Commonly Available Clinical Biomarkers

Currently, colonoscopy is the most reliable method available for early detection of CRC and its precursors. However, many patients hesitate or completely avoid having a colonoscopy due to its cost and invasive nature. The fecal occult blood test (FOBT), which is the most widely used noninvasive screening tool currently used, is limited by its low sensitivity, especially with respect to detection of pre-neoplastic lesions. Stool DNA tests may be a promising alternative in the future, but widespread application is limited by labor-intensive handling and high costs. Alternatively, current blood biomarkers such as carcinoembryonic antigen (CEA) have less-than-ideal sensitivity and specificity.

The miRNA markers described herein in tissue and plasma have shown superior sensitivity and specificity values as compared to other reported miRNAs (e.g., miR-29a, miR-92, and miR-221) and commonly available clinical biomarkers such as CEA, PSA, immunochemical fecal occult blood test (iFOBT), and guaiac based FOBT (gFOBT) (Table 3).

TABLE 3
Comparison of our current miRNA panel and plasma marker to other
circulatory miRNAs and commonly used clinical biomarkers

| | | Area | | |
|---|---|---|---|---|
| | | Under | | |
| | Normal | the | Sensi- | Speci- |
| | Range | Curve | tivity | ficity |
| Markers | (ng/ml) | (AUC) | (%) | (%) |
| Carcinoembryonic Antigen (CEA) | <2.5 | 0.808 | 36 | 87 |
| Fakih et al., Oncology 2006 | <2.5 | | 74 | 83 |
| Immunochemical Fecal Occult | Not | Not | 67 | 85 |
| Blood Test (iFOBT) | appli- | calcu- | | |
| Zhu et al., Journal of Digestive | cable | lated | | |
| Diseases 2011 | | | | |
| Guaiac Fecal Occult Blood Test | | | 54 | 80 |
| (gFOBT) | | | | |
| Zhu et al., Journal of Digestive | | | | |
| Diseases 2011 | | | | |
| miR-92 | Not | 0.855 | 89 | 70 |
| Ng et al., Gut 2009 | appli- | | | |
| miR-29a | cable | 0.844 | 83 | 85 |
| Huang et al., International | | | | |
| Journal of Cancer 2010 | | | | |
| miR-221 | | 0.606 | 86 | 41 |
| Pu et al., Journal of | | | | |
| Gastroenterology and | | | | |
| Hepatology 2011 | | | | |
| Four-microRNA tissue panel | Not | 0.932 | 100 | 85 |
| Plasma miR-21 | appli- | 0.910 | 90 | 90 |
| | cable | | | |

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

APPENDIX I
Demographics of enrolled patients

| | | | | | Comparison Group | |
|---|---|---|---|---|---|---|
| | Tissue Group | | Plasma Group | | (CG) | |
| | Training* | Test | Training* | Test | CG1^{§} | CG2^{¶} |
| Subtype | (n = 30) | (n = 16) | (n = 30) | (n = 20) | (n = 30) | (n = 20) |

Gender

| | | | | | | |
|---|---|---|---|---|---|---|
| Men (n, %) | 14 (47) | 10 (63) | 14 (53) | 7 (35) | 14 (47) | 7 (35) |
| Women (n, %) | 16 (53) | 6 (37) | 16 (47) | 13 (65) | 16 (53) | 13 (65) |

Age at Diagnosis

| | | | | | | |
|---|---|---|---|---|---|---|
| Mean ± SD | 60 ± 11 | 66 ± 13 | 60 ± 11 | 57 ± 11 | 61 ± 9 | 58 ± 9 |
| Median (Range) | 60 (43-76) | 66 (48-92) | 60 (43-76) | 58 (39-78) | 60 (42-78) | 58 (43-74) |

TNM Stage

| | | | | | |
|---|---|---|---|---|---|
| I | 3 | 0 | 3 | 0 | Not applicable |
| II | 4 | 5 | 4 | 5 | |
| III | 15 | 3 | 15 | 8 | |
| IV | 4 | 1 | 4 | 7 | |
| Not staged | 4 | 7 | 4 | 0 | |

Tumor Location

| | | | | | |
|---|---|---|---|---|---|
| Colon | 30 | 11 | 30 | 12 | Not applicable |
| Rectum | 0 | 5 | 0 | 8 | |

*The same patients were used in the training group for the tissue and plasma studies
^{§}CG1 was used as a match comparison group with the plasma training group
^{¶}CG2 was used as a race-, gender-, and age-match comparison group with the plasma test group

1. (canceled)

2. An article of manufacture, comprising:
a pair of miR-21 amplification primers; and
a pair of U6B amplification primers.

3. The article of manufacture of claim 2, further comprising
instructions for amplification conditions for determining the level of miR-21 in a biological sample;
instructions for using plasma as the biological sample; and
instructions for determining the presence of sporadic CRC in the individual, wherein a higher level of miR-21 relative to the level of miR-21 in a control sample is indicative of the presence of sporadic CRC in the individual.

4. An article of manufacture, comprising:
a pair of miR-31 amplification primers;
a pair of miR-135b amplification primers;
a pair of miR-1 amplification primers;
a pair of miR-133a amplification primers; and
a pair of U6B amplification primers.

5. The article of manufacture of claim 2, further comprising
instructions for amplification conditions for determining the levels of at least four miRNAs in a biological sample;
instructions for using colon or rectal tissue as the biological sample; and
instructions for determining the presence of sporadic CRC in the individual, wherein a higher level of miR-31 and miR-135b and a lower level of miR-1 and miR-133a relative to corresponding miRNA levels in a control sample is indicative of the presence of sporadic CRC in the individual.
